(19) United States
(12) Patent Application Publication
(10) Pub. No.: US 2014/0242542 A1
(43) Pub. Date: Aug. 28, 2014

(54) Dental Handpiece Rack and Method of Storing Dental Handpieces

(71) Applicant: Jason Jubenville, Duncan (CA)

(72) Inventor: Jason Jubenville, Duncan (CA)

(21) Appl. No.: 14/085,456

(22) Filed: Nov. 20, 2013

Related U.S. Application Data
(60) Provisional application No. 61/769,550, filed on Feb. 26, 2013.

(51) Int. Cl. A61G 15/16; A61L 2/26; A61B 19/00

(52) U.S. Cl. CPC A61G 15/16; A61B 19/44; A61L 2/26; A61B 2019/446
USPC 433/79; 433/77

(57) ABSTRACT

A dental handpiece and general implement rack is provided, along with a method of tracking implement use in a patient environment. The rack comprises a housing having a plurality of removable trays therein, the trays being adapted to support a dental implement thereon. Each tray includes a visual indicator that matches a marker placed on an implement being stored in that tray. The tray indicators are utilized to determine which implement has been utilized during the course of the day in order to implement sterilization procedures for only those implements that have been used on patients and are now unsterile. The device and associated method prevent sterilization by autoclave for implements that do not require cleaning, thereby extending their useful life and preventing over-cleaning thereof. The housing is preferably a wall-mountable structure having a hinged lid closure and a ventilated design to prevent moisture capture therein.

### CROSS REFERENCE TO RELATED APPLICATION

This application claims the benefit of U.S. Provisional Application No. 61/769,550 filed on Feb. 26, 2013, entitled “Rack.” The above identified patent application is herein incorporated by reference in its entirety to provide continuity of disclosure.

### BACKGROUND OF THE INVENTION

1. Field of the Invention

The present invention relates to tool supports and storage racks. More specifically, the present invention pertains to a new and novel dental handpiece support for organizationally storing and tracking dental handpieces, thereby preventing inadvertent sterilization of unused handpieces and unnecessary wear thereof.

Dental handpieces, or dentist's drills, are high speed drill tools utilized during dental procedures for removing enamel and reshaping teeth. These devices include air driven or electrically driven drill devices having different bit attachments capable various drilling operations. These devices generally operate at very high rotational speeds and are critical tools for dental procedures.

Dental handpieces are required to be sterilized between patients in order to prevent cross contamination from one patient to another. However, because of the sensitive design and components of a handpiece, they cannot be simply placed into an alcohol bath for sterilization. Rather, these devices must be placed into an autoclave after being hand cleaned and lubricated. The autoclave kills any germs or bacteria that may cross between patients in subsequent uses of the device, and prevents damage thereto that would otherwise result from complete emersion or use of alcohol as a cleaning solution.

In order to accommodate many patients throughout the day, most dentists employ several handpieces in an office and switch from a used handpiece to one that is clean and ready for use from the autoclave when treating a new patient. While this adds cost to the practice, it maintains a sterile tool for each patient and allows time for the autoclave to sterilize each handpiece after a given use. One common issue with this practice is ensuring only used handpieces are sterilized, and those that may not have been used during the day are not subjected to the autoclave environment unnecessarily.

Subjecting a sophisticated tool such as a dentist's handpiece to an autoclave environment invariably causes wear on the moving components of the device and reduces its useful life. Eventually the handpiece will not lubricate properly or components will become physically altered over time. This is normal and requires the dental professional to either have the handpiece repaired or replaced with a replacement article. However, it is a goal of every business to prevent waste and to prevent unnecessary expenditures, therefore maintaining dental tools and preventing premature replacement or repair is highly desired.

The present invention provides an organizational tool and method that prevents an unused handpiece from being inadvertently sanitized without first being used, whereby a plurality of handpieces are supported in individually identifiable trays that allow dental professionals to recognize used and unused handpieces. The device comprises a storage rack having a plurality of removable trays that are each adapted to support a single dental handpiece thereon. Deployed on each handpiece is a marker that corresponds with a given rack, whereby the dental workers can identify those handpieces that have been used and those that haven't by color order, preventing one handpiece from being re-used and preventing unnecessary sanitizing procedures from being employed on non-used handpieces by mistake.

2. Description of the Prior Art

Devices have been disclosed in the prior art that relate to storage devices and storage racks for tools. These include devices that have been patented and published in patent application publications. These devices relate to general storage devices or specific supports for dental tools. The following is a list of devices deemed most relevant to the present disclosure, which are herein described for the purposes of highlighting and differentiating the unique aspects of the present invention, and further highlighting the drawbacks existing in the prior art.

Devices in the prior art relate to dental appliance supports and dental tool supports. The devices describe assemblies that support various dental tools for improved access thereto and for reduced clutter in a dental professional workspace. These include various handpiece supports, caddies, racks, and organizational tables that allow the doctor or hygienist to quickly grasp a tool when required and organize its storage when not in use. Most devices relate to basic supports for various instruments and not to any improved method of sterilization tracking. The present invention relates to a handpiece support rack that allows a dental professional to recognize used and unused handpieces at first glance, preventing unnecessary and harmful cleaning procedures on otherwise clean and ready handpieces.

Examples of devices in the prior art include U.S. Pat. No. 5,332,392 to Bierbaum, which discloses a dental instrument deposit table that includes a support surface and end holders for supporting handpieces therefrom. Another device related in the art is U.S. Pat. No. 4,571,182 to Beier, which discloses a dental implement carrying plate having positioning elements extending therefrom that support the electrical, fluid or pneumatic connections to the dental implements. Finally, several designs exist in the art that relate to dental trays and supports for dental implements. These include U.S. Design Pat. No. D269,396 to Bricker, U.S. Pat. No. D261,424 to Anson, and U.S. Pat. No. D293,371 to Nordstrom. While disclosing novel designs for implement supports and caddies therefor, these designs do not disclose the functional aspects of the present handpiece tray.

The present invention provides a new dental implement rack that is particularly suited for supporting dental handpieces and preserving the same. The rack includes a plurality of removable and color-coded trays, whereby the tray color corresponds with a colored marker of a supported handpiece. The dental professional uses the color markings to visually track which handpieces are used throughout the day and which ones need to be sterilized before being re-used. This allows the professional to avoid excessive cleaning of handpieces, which is detrimental to their operation in the long-term and an unnecessary expenditure. Further disclosed is an accompanying method of storing dental handpieces using the present device to prevent unnecessary autoclave sterilization.

It is submitted that the present invention is substantially divergent in design elements from the prior art, and consequently it is clear that there is a need in the art for an improvement to existing dental implement support devices. In this regard the instant invention substantially fulfills these needs.

### SUMMARY OF THE INVENTION

In view of the foregoing disadvantages inherent in the known types of dental implement supports now present in the prior art, the present invention provides a new dental handpiece and general dental implement support, wherein the same can be utilized for providing convenience for the user when storing dental implements and tracking those that require sterilization by autoclave.

It is therefore an object of the present invention to provide a new and improved dental handpiece support device that has all of the advantages of the prior art and none of the disadvantages.

It is another object of the present invention to provide a dental handpiece support device that utilizes removable trays having a color-coded system thereon, whereby the color of the tray is coordinated with a marker placed on the end of the dental handpiece stored within the tray interior.

Another object of the present invention is to provide a dental handpiece support device that reduces or eliminates the probability that a handpiece will be sterilized by autoclave more than once between uses, thereby extending the useful life of the tool and saving the dental professional unnecessary replacement or repair costs.

Another object of the present invention is to provide a dental handpiece support device that extends the useful life of existing and new dental handpieces by preventing overuse of sterilization procedures thereon.

Another object of the present invention is to provide a dental handpiece support device that may comprise a housing that is wall-mountable, wherein the trays can be accessed by a dental professional while in a seated or standing position for retrieving a handpiece.

Another object of the present invention is to provide a dental handpiece support device that provides an outer cover and ventilation means for the trays and the cover, whereby moisture is not trapped within the housing.

Yet another object of the present invention is to provide a dental handpiece support device that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the present invention in a working state with its hinged door in an open state, along with a close-up view of the contemplated marker applied to each handpiece.

FIG. 2 shows a view of the removable trays of the present invention.

FIG. 3 shows a side view of the removable trays of the present invention.

FIG. 4 shows a view of the dental handpieces being supported within the removable trays of the present invention.

### DETAILED DESCRIPTION OF THE INVENTION

Reference is made herein to the attached drawings. Like reference numerals are used throughout the drawings to depict like or similar elements of the present dental handpiece support. For the purposes of presenting a brief and clear description of the present invention, the preferred embodiment will be discussed as used for supporting dental handpieces and other implements, and for notifying the user which implements require sterilization and which of those do not. The figures are intended for representative purposes only and should not be considered to be limiting in any respect.

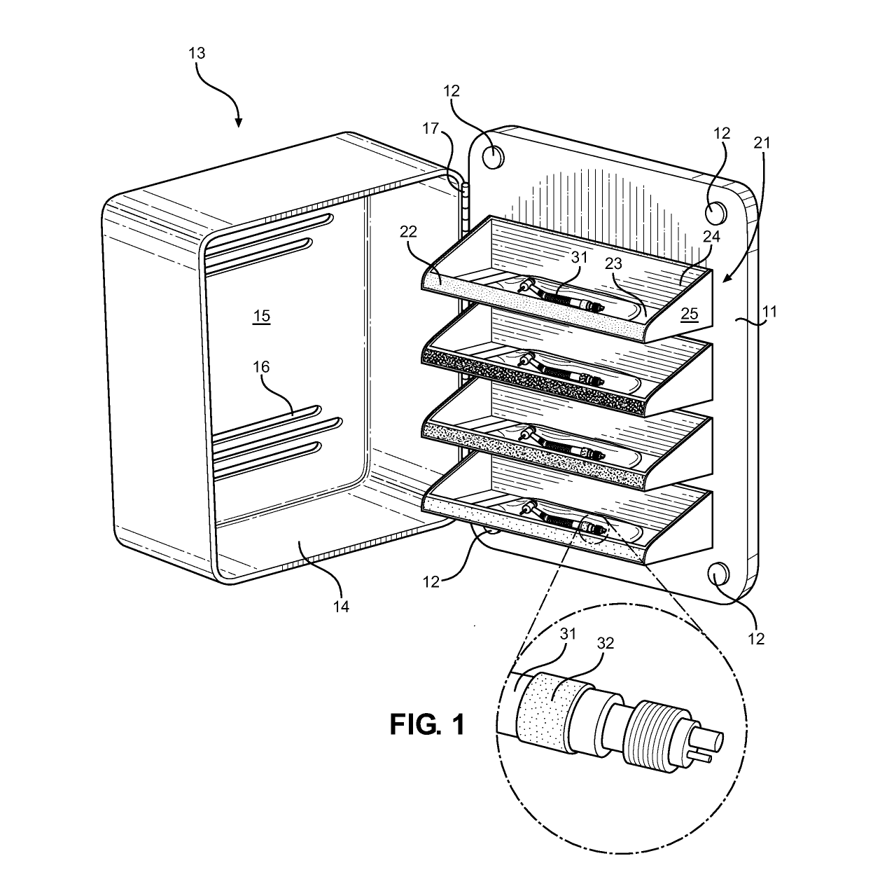

Referring now to FIG. 1, there is shown a perspective view of the dental handpiece support of the present invention. The support comprises a rear surface 11 supporting a plurality of removably attached implement support trays 21 thereon. The support trays 21 are aligned vertically and disposed along the rear surface 11 in a manner that promotes organization and orderly storage of handpieces 31 within the assembly. Each tray 21 supports an individual handpiece before and after use, whereby each tray includes an outer lip surface 22 having a defined color or pattern that is adapted to match a marker element 32 placed on the proximal end of each handpiece 31. In this way, each handpiece 31 has a defined tray 21 location within which it is supported and the user can tray which handpiece has been utilized during the day to ensure sterilization of only those handpieces 31 that have been in contact with a patient.

Each tray 21 comprises a base surface 23, an upstanding back wall 24, a pair of lateral sidewalls 25 that connect the base surface 23 with the upstanding back wall 24. The base surface 23 forms an interior area within which to place a handpiece 31 therein, while the back wall 25 and side walls 25 retain the handpiece 31 therein. Along the outer edge of the base surface 25 is an upstanding outer lip surface 22, which comprises a colored or patterned surface that matches the marker 32 placed on the handpiece 31 within that tray interior. During operation, the dental professional uses one handpiece 31 at a time and replaces the used handpiece 31 in the tray 21 after use. At the end of the day, the handpieces 31 are organized within the present assembly and can easily be gathered and placed into an autoclave for sterilization thereof.

To secure the trays 21, each is removably affixed to the rear surface 11 of the assembly. The rear surface 11 is preferably wall-mountable, wherein a plurality of mechanical fasteners 12 is deployed through a support wall to support the rear surface 11 thereon and within an office. It is desired to provide a hinged closure door 13 attached to the rear surface 11, which can secure over the trays 21 and shield them from view until a dental professional requires the tool for use or access for replacement thereof on a tray 21. The door 13 is attached to the rear surface 11 along one edge thereof and by way of a hinge joint 17, which allows the door 13 to swing from an open position to a closed position over the trays 21 while the trays 21 are attached to the rear surface 11. In an alternate embodiment of the present invention, the trays 21 may be permanently affixed to the rear surface 11, as opposed to being removably attached.

The rear surface 11 and the hinged door 13 combine to form a securable housing, wherein the door 13 comprises extended sidewalls 14 and an outer surface 15 to enclose the trays 21 therein and secure against the outer edges of the rear surface 11. To allow air flow into the housing when the door 13 is closed, a plurality of slits 16 or apertures may be disposed along the outer surface 15 or sidewalls 14 of the door 13. This prevents moisture build-up therein.

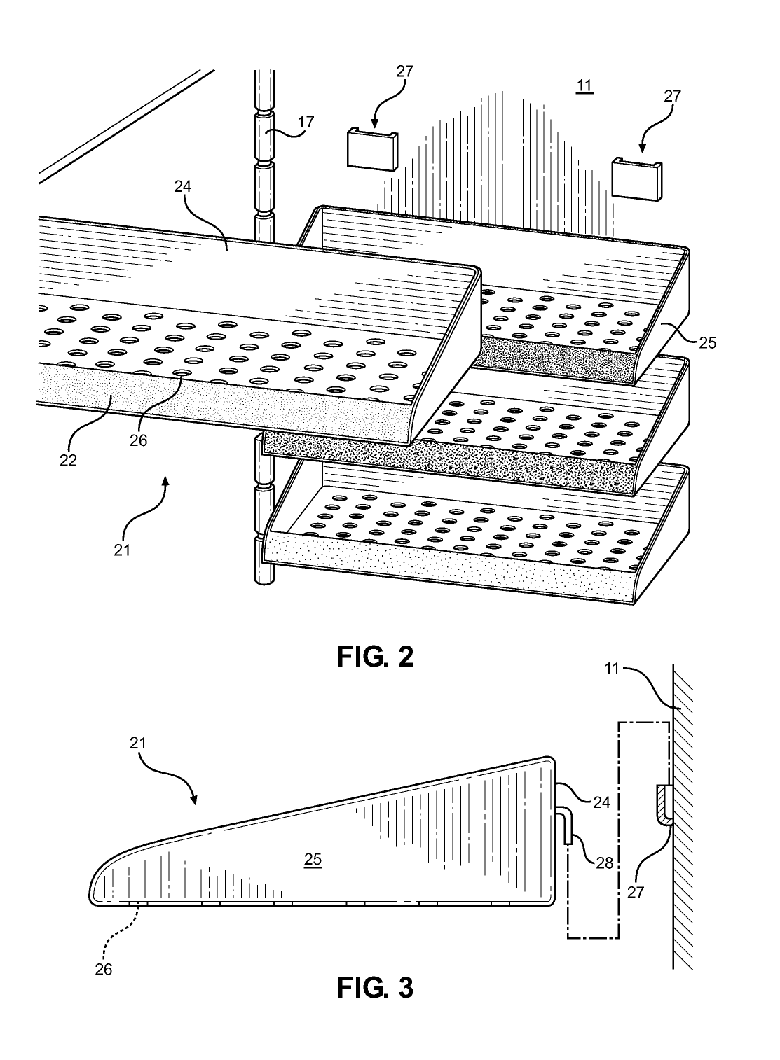

Referring now to FIGS. 2 and 3, there are shown views of the trays 21 of the present invention and their color-coded outer lip surface 22, along with the contemplated attachment means to the rear surface 11. Each tray 21 includes an upstanding back wall 24 that is adapted to abut against the rear surface 11 of the assembly housing and connect thereto. To secure the back wall 24 of each tray 21 to the rear surface 11, a first and second connector 27 is provided along the rear surface 11. The connectors 27 comprise a first and second enclosed ledge or aperture that is adapted to accept a downward directed clip 28 extending outward from the tray back wall. The clip 28 secures within the connector 27 interior, which supports the tray 21 and any contents therein in a cantilevered configuration from the rear surface 11.

To improve drainage and moisture evaporation from within the trays 21 and from an implement stored therein, the tray base surface 26 may comprise a plurality of drainage apertures 26 adapted to allow air flow into the tray interior. This allows implements to air dry therein and not pool standing water in the tray after a procedure with the implement. To access the trays 21, the user can simply access the tray interior directly, or if the outer door is closed, the user can rotate the door into an open configuration by way of the hinge joint 17 and access the trays 21. Each tray can be then removed 21 or retained within the housing, and the implement therein can be withdrawn or replaced therein as necessary. The marker placed on each implement (handpiece or similar dental tool) is adapted to correspond with a given tray outer lip surface color, whereby tool tracking is facilitated.

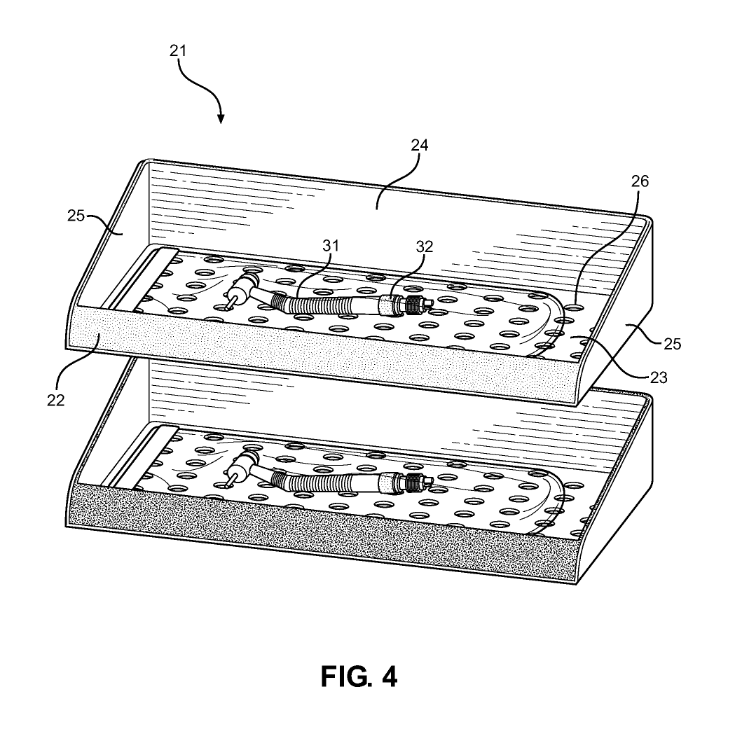

Referring finally to FIG. 4, there is shown a perspective view of two trays 21 of the present invention, wherein each tray is supporting a dental handpiece 31 having a defined marker 32 attached thereto. Along with the apparatus of the present invention, it is desired to disclose a method of tool tracking and method of preventing over-sterilization of handpieces in a dental office environment. The method steps include marking a handpiece 31 with a color-coded marker element 32 along its proximal end, and storing the handpiece in a tray 21 for later use. The tray 21 supporting the handpiece includes an outer surface lip 22 that is adapted to match the color-coded marker element 32 of the handpiece 31. A dental professional can access the handpiece 31 and use it in a dental procedure, after which the handpiece 31 is replaced on the corresponding tray 21 matching its attached marker 32. At the end of the day, a user can visualize those trays that have been utilized and the corresponding handpiece that has been in contact with a patient, whereby those used handpieces require sterilization by autoclave and those not used do not. During storage, the side walls 25, back wall 24, base surface 23, and outer surface lip 22 retain the implement in a ready to use configuration that promotes organization and clean storage thereof.

When operating a dental handpiece or similar dental implement, it is important to sterilize each hand piece the same number of times and not subject the implement to excessive and unnecessary cleanings. When one hand piece gets sterilized more than the others, it wears out and needs to be repaired or replaced more frequently and quickly than necessary, which becomes expensive and frustrating. However, keeping track of which have been used can be time consuming and tedious. Along with tool tracking, these items need to be readily accessible for use on patients in a dental office environment.

The present invention therefore describes a storage assembly and tool tracking means for dental handpieces. The assembly comprises a housing for supporting and storing handpiece support trays. The trays can be removed for moving the implement carried therein, or for general cleaning or repair thereof. The device provides a means of organizing dental handpieces, and provides a secure storage location that promotes organization and office efficiency. The dental handpieces can be tracked if they are removed or the entire tray is removed such that each item is used and sterilized the same number of times to reduce damage and wear to the implement, and therefore reduce unnecessary replacement or repair cost to the owner.

It is submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A dental implement storage and organization device, comprising:
a rear surface;
a plurality of dental implement support trays attached to said rear surface;
said dental implement support trays having an upstanding back wall, a base surface, a pair of lateral side walls connecting said back wall and base surface, and an outer lip surface;
each tray having an area adapted to store an individual dental implement therein;
said outer lip surface of each tray having a surface design adapted to match a marker element placed on a dental implement stored therein.

2) The dental implement storage and organization device of claim 1, wherein said base surface of each tray further comprises a plurality of apertures thereon.

3) The dental implement storage and organization device of claim 1, wherein said trays are removably attached to said rear surface.

4) The dental implement storage and organization device of claim 1, wherein:
said trays are removably attached to said rear surface;
said trays further comprise a first and second downward directed clip extending from said back wall;
said rear surface having a plurality of horizontally aligned first and second clip connectors adapted to accept said first and second downward directed clip of each tray.

5) The dental implement storage and organization device of claim 1, further comprising:
a door connected to an edge of said rear surface by a hinge joint;
said door comprising an outer surface adapted to match said rear surface area, sidewalls extending from said door outer surface;
said outer surface and sidewalls forming an open door interior adapted to accept said trays therein when said door is in a closed position against said rear surface.

6) The dental implement storage and organization device of claim 5, wherein:
said outer surface of said door further comprises apertures therethrough.

7) The dental implement storage and organization device of claim 1, wherein:
said plurality of trays is disposed in vertical alignment along said rear surface.

8) The dental implement storage and organization device of claim 1, wherein:
said rear surface is wall-mountable.

9) The dental implement storage and organization device of claim 1, wherein:
said surface design of said outer lip surface further comprises a specific color.

10) The dental implement storage and organization device of claim 1, wherein:
said surface design of said outer lip surface further comprises a specific indicia.

11) The dental implement storage and organization device of claim 1, wherein:
said surface design of said outer lip surface further comprises a specific pattern.

12) A method of tracking dental appliances and ensuring sterilization of each appliance occurs only once after each use using the dental implement storage and organization device of claim 1, comprising the steps of:
placing a single dental implement in a designated tray;
marking said single dental implement with a unique marker having a design adapted to match said outer surface lip design of said designated tray;
maintaining said dental implement within said designated tray having matching marker and outer surface lip design between uses;
sterilizing those implements stored within those trays having used implements;
recognizing those implements not requiring sterilization within trays having unused implements.
